(19) United States
(12) Patent Application Publication
(10) Pub. No.: US 2023/0012051 A1
(43) Pub. Date: Jan. 12, 2023

(54) PATIENT INFORMATION PRESENTATION METHOD AND ELECTRONIC DEVICE

(71) Applicant: Wistron Corporation, New Taipei City (TW)

(72) Inventor: TE-AN CHANG, New Taipei City (TW)

(73) Assignee: Wistron Corporation, New Taipei City (TW)

(21) Appl. No.: 17/489,797

(22) Filed: Sep. 30, 2021

(30) Foreign Application Priority Data
Jul. 6, 2021 (TW) 110124709

(51) Int. Cl. G16H 10/60; G06F 3/0485; G06F 3/0484; G16H 50/30

(52) U.S. Cl. CPC G16H 10/60; G06F 3/0485; G06F 3/04845; G16H 50/30

(57) ABSTRACT

The disclosure provides a patient information presentation method and an electronic device, which allows users to configure the content, size and layout of each patient information card in the browsing interface according to their needs, so that the users can view various patient information in their own way.

### CROSS-REFERENCE TO RELATED APPLICATION

This application claims the priority benefit of Taiwan application serial no. 110124709 filed on Jul. 6, 2021. The entirety of the above-mentioned patent application is hereby incorporated by reference herein and made a part of this specification.

### BACKGROUND

### Technical Field

This disclosure relates to an information presentation technology, and in particular to a patient information presentation method and an electronic device.

### Description of Related Art

In the related art, in order to allow medical staff to quickly access cross-system clinical items, some hospitals have provided a cross-system integrated data framework, while also establishing a data presentation dashboard for reference by a doctor.

In general, most of the aforementioned hospitals use a fixed combination of clinical items and dashboards to provide patient information inquiry by different specialties and different doctors. Namely, the related art lacks an inquiry system capable of flexibly combining clinical data of the different specialties and patient characteristics, and also lacks the feasibility of flexibly expanding dashboard data.

### SUMMARY

This disclosure provides a patient information presentation method and an electronic device, which are used to resolve the aforementioned technical problem.

An embodiment of the disclosure provides a patient information presentation method adapted to an electronic device, and includes the following steps. A user interface is switched to an editing interface in response to determining that an edit button in the user interface is triggered, in which the editing interface includes a first wallpaper and multiple patient information cards. A first patient information card is pinned to a first position on the first wallpaper in a corresponding first predetermined size in response to determining that the first patient information card in the multiple patient information cards is dragged to and placed at the first position on the first wallpaper. And, the user interface is switched to a browsing interface in response to determining that an end editing button in the editing interface is triggered, and the first wallpaper is presented in the browsing interface, in which the first wallpaper includes the first patient information card set in the first position, and the first patient information card presents information corresponding to a current patient of interest.

An embodiment of the disclosure provides an electronic device, which includes a display, a storage circuit, an input interface, and a processor. The display displays a user interface. The storage circuit stores a program code. The input interface receives a trigger event executed on the user interface. The processor is coupled to the display, the storage circuit and the input interface, and accesses the program code to execute the following steps. The user interface is switched to an editing interface in response to determining that an edit button in the user interface is triggered, in which the editing interface includes a first wallpaper and multiple patient information cards. A first patient information card is pinned to a first position on the first wallpaper in a corresponding first predetermined size in response to determining that the first patient information card in the multiple patient information cards is dragged to and placed at the first position on the first wallpaper. And, the user interface is switched to a browsing interface in response to determining that an end editing button in the editing interface is triggered, and the first wallpaper is presented in the browsing interface, in which the first wallpaper includes the first patient information card set in the first position, and the first patient information card presents information corresponding to a current patient of interest.

To make the abovementioned more comprehensible, several embodiments accompanied by drawings are described in detail as follows.

### BRIEF DESCRIPTION OF THE DRAWINGS

FIG. 1 is a schematic diagram of an electronic device according to an embodiment of the disclosure.

FIG. 2 is a flowchart of a patient information presentation method according to an embodiment of the disclosure.

FIG. 3A is a schematic diagram of a user interface according to an embodiment of the disclosure.

FIG. 3B is a schematic diagram of an editing interface according to FIG. 3A.

FIG. 3C is a schematic diagram of presenting patient information cards in an item selection box according to FIG. 3B.

FIGS. 3D to 3G are schematic diagrams of various patient information cards corresponding to different patient information items according to an embodiment of the disclosure.

FIG. 4A is a schematic diagram showing multiple patient information cards according to an embodiment of the disclosure.

FIG. 4B is a schematic diagram of pinning a patient information card according to FIG. 4A.

FIG. 4C is a schematic diagram of finishing editing the first wallpaper according to FIG. 4B.

FIG. 4D is a schematic diagram of a browsing interface according to FIG. 4C.

FIG. 5 is a schematic diagram of a saved template according to FIG. 4C.

FIG. 6A is a schematic diagram of applying a for personal use template according to an embodiment of the disclosure.

FIG. 6B is a schematic diagram of applying a for public use template according to FIG. 6A.

FIG. 6C is a schematic diagram of applying a for public use by unit template according to FIG. 6A.

FIG. 7A is a schematic diagram of a newly added page according to an embodiment of the disclosure.

FIG. 7B is a schematic diagram of switching pages according to FIG. 7A.

FIG. 8 is a schematic diagram of expanding a patient button field according to FIG. 4D.

FIG. 9A is a schematic diagram of ward switching according to an embodiment of the disclosure.

FIG. 9B is a schematic diagram of another browsing interface according to FIG. 9A.

FIG. 10 is a schematic diagram of patient information cards with different predetermined sizes according to an embodiment of the disclosure.

FIGS. 11A to 11D are schematic diagrams of adjusting sizes of patient information cards according to different embodiments of the disclosure.

FIGS. 12A to 12C are schematic diagrams of patient information cards of a warning type according to different embodiments of the disclosure.

### DESCRIPTION OF THE EMBODIMENTS

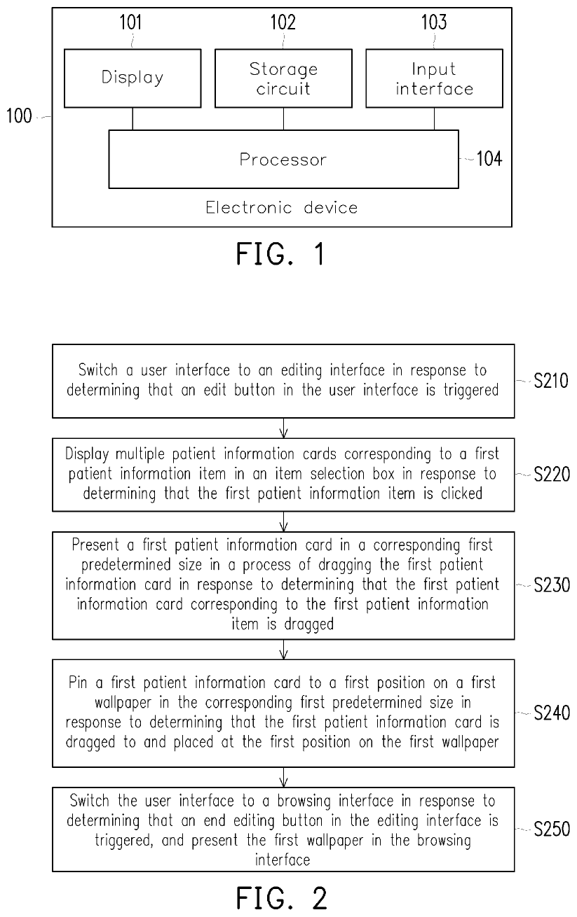

Referring to FIG. 1, FIG. 1 is a schematic diagram of an electronic device according to an embodiment of the disclosure. In different embodiments, the electronic device 100 is, for example, various computer devices and/or smart devices, but the disclosure is not limited thereto. In an embodiment, the electronic device 100 is, for example, a hospital information system (HIS) computer that may be used by doctors in a hospital to inquire patient information in each inpatient ward, but the disclosure is not limited thereto.

As shown in FIG. 1, the electronic device 100 includes a display 101, a storage circuit 102, an input interface 103, and a processor 104. The display 101 may be used to display a user interface. The storage circuit 102 is, for example, any type of a fixed or removable random access memory (RAM), a read-only memory (ROM), a flash memory, a hard disk or other similar devices or a combination of these devices that may be used to record multiple program codes or modules. The input interface 103 is, for example, a mouse, a keyboard, or other devices that may be used to receive a trigger event executed on the aforementioned user interface. In some embodiments, the display 101 and the input interface 103 may be integrated into a touch screen, but the disclosure is not limited thereto.

The processor 104 is coupled to the display 101, the storage circuit 102, and the input interface 103, and may be a general-purpose processor, a special-purpose processor, a conventional processor, a digital signal processor, and multiple microprocessors, one or more microprocessors combined with a digital signal processor kernel, a controller, a microcontroller, an application specific integrated circuit (ASIC), a field programmable gate array (FPGA), any other types of integrated circuit, a state machine, a processor based on advanced RISC machine (ARM) and similar products.

In an embodiment of the disclosure, the processor 104 may access modules and program codes recorded in the storage circuit 102 to implement the patient information presentation method of the disclosure, and details thereof are described as follows.

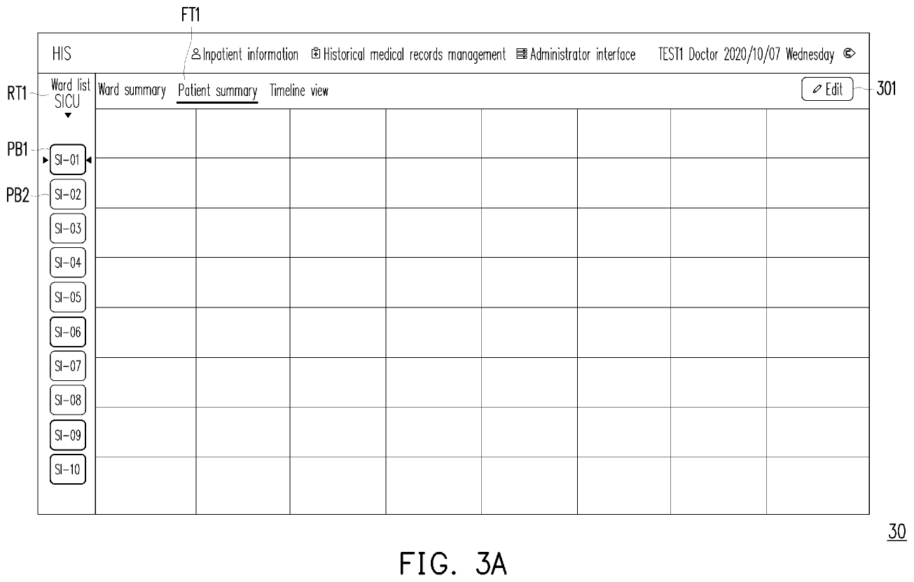

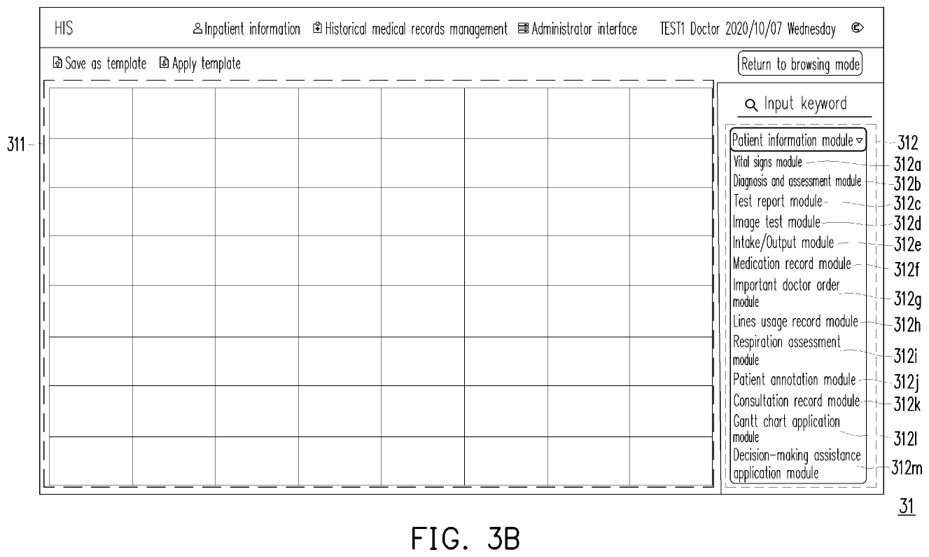

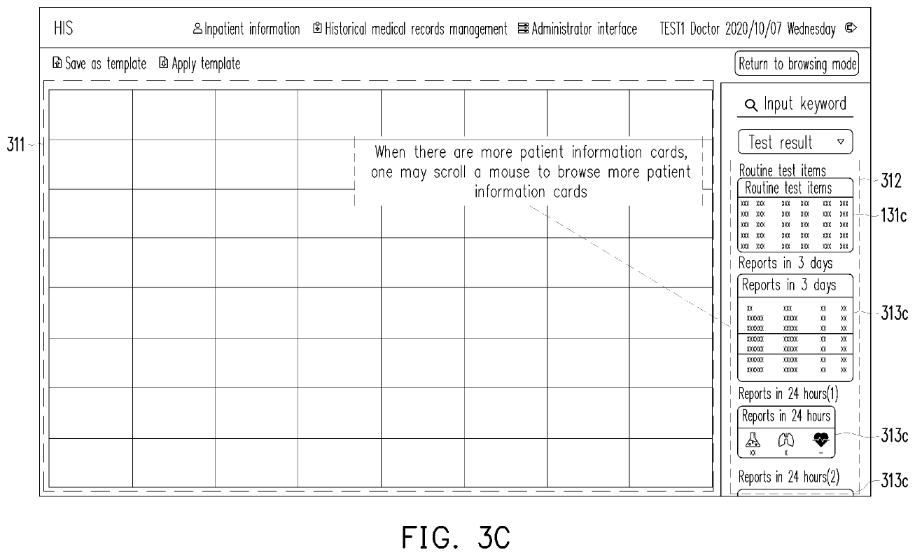

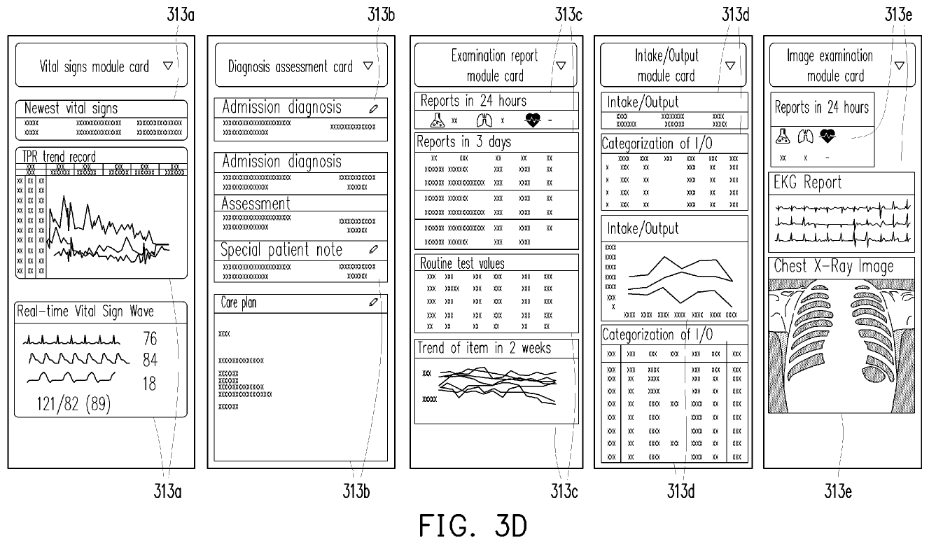

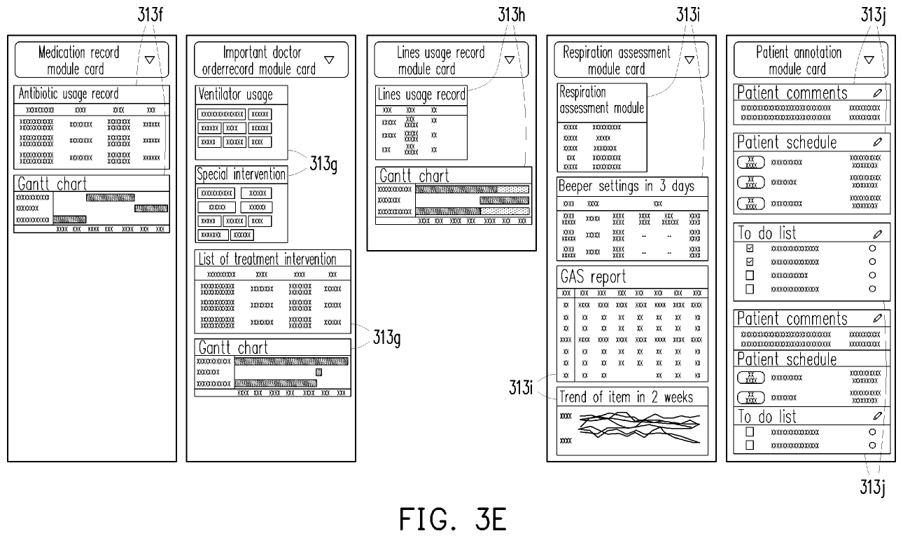

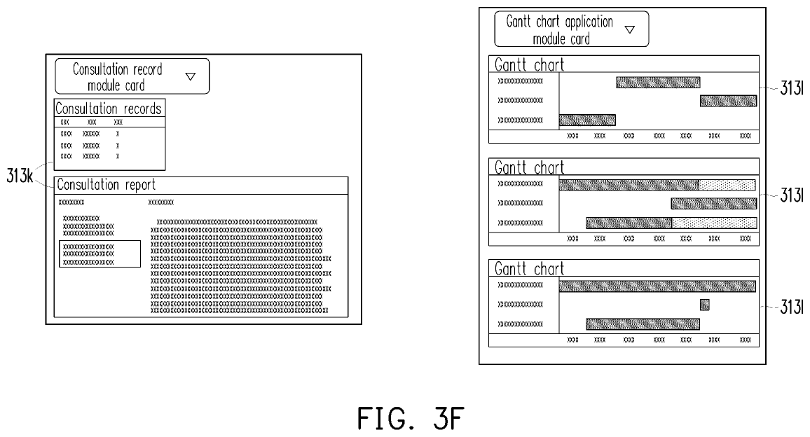

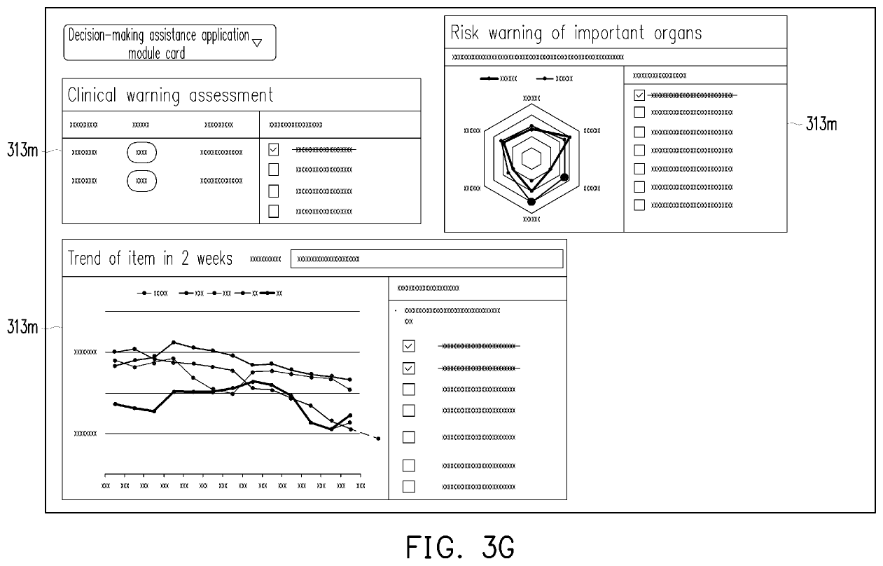

Referring to FIG. 2, FIG. 2 is a flowchart of a patient information presentation method according to an embodiment of the disclosure. The method of the embodiment may be executed by the electronic device 100 in FIG. 1, and details of each step in FIG. 2 will be described below with reference of the components shown in FIG. 1. In addition, in order to make the technology of the disclosure easier to understand, FIG. 3A is provided below for further explanation.

Referring to FIG. 3A, FIG. 3A is a schematic diagram of a user interface according to an embodiment of the disclosure. In an embodiment, the processor 104 may execute the aforementioned HIS, and after a user (such as a doctor) logs in the aforementioned HIS, the processor 104 may, for example, control the display 101 to provide a user interface 30 shown in FIG. 3A (which is, for example, a webpage interface).

In FIG. 3A, the user interface 30 may now be presented as a dashboard interface (which may also be referred to as a browsing interface). As shown in FIG. 3A, the user interface 30 may include various function tags (such as a ward summary tag, a patient summary tag FT1, etc.), a ward menu button RT1, and multiple patient buttons (such as patient buttons PB1 and PB2). In FIG. 3A, the ward menu button RT1 may be used to allow the user to select a ward of interest, such as a surgical intensive care unit (SICU) shown in FIG. 3A. In addition, the multiple patient buttons shown in FIG. 3A may respectively correspond to multiple patients belonging to the SICU, and referential numbers thereof are, for example, SI-01 to SI-10, but the disclosure is not limited thereto.

In the embodiment of the disclosure, the electronic device 100 allows the user to customize the browsing interface required when accessing the patient summary tag FT1 in the HIS, and the browsing interface may include cards used for presenting various patient information. In this way, the user may place the aforementioned cards at any position in the browsing interface according to their own needs, thereby improving the user's experience in finding patient information.

In the situation of FIG. 3A, it is assumed that the user has not made any edit to the required browsing interface. Therefore, the browsing interface is currently illustrated as a blank wallpaper, but the disclosure is not limited thereto. In an embodiment, when the user wants to edit the browsing interface, the user may click/trigger an edit button 301 in the user interface 30 through the input interface 103.

Correspondingly, in step S210, in response to determining that the edit button 301 in the user interface 30 is triggered, the processor 104 switches the user interface 30 to an editing interface 31, as shown in FIG. 3B.

Referring to FIG. 3B, FIG. 3B is a schematic diagram of the editing interface according to FIG. 3A. In FIG. 3B, the editing interface 31 may include a first wallpaper 311 and an item selection box 312. The item selection box 312 includes multiple patient information items 312a to 312m. In different embodiments, the patient information items 312a to 312m may include at least one of vital signs information, diagnosis and annotation information, examination result information, imaging examination information, intake/output record information, medication record information, doctor order information, vascular lines record information, consultation record information and ventilator record information, but the disclosure is not limited thereto.

In different embodiments, the first wallpaper 311 may include N×M grids, where N and M are positive integers. In FIG. 3B, the first wallpaper 311 includes, for example, 8×8 grids, but the disclosure is not limited thereto.

In the embodiment of the disclosure, when any of the patient information items 312a to 312m (which are referred to as first patient information items hereinafter) is selected, the item selection box 312 may display multiple patient information cards belonging to the selected first patient information item for being dragged to a desired position on the first wallpaper 311 by the user for placement.

In an embodiment, it is assumed that the selected first patient information item is the patient information item 312c in FIG. 3B, in step S220, in response to determining that the first patient information item is selected, the processor 104 may display multiple patient information cards corresponding to the first patient information item in the item selection box 312, as shown in FIG. 3C.

Referring to FIG. 3C, FIG. 3C is a schematic diagram of presenting the patient information cards in the item selection box according to FIG. 3B. In FIG. 3C, the item selection box 312 may display multiple patient information cards 313c corresponding to the patient information item 312c. In an embodiment, when the number of the patient information cards corresponding to the first patient information item is larger, the user may browse more patient information cards by scrolling a mouse, but the disclosure is not limited thereto.

In different embodiments, the patient information cards corresponding to different patient information items may have different forms, as shown in FIGS. 3D to 3G.

Referring to FIGS. 3D to 3G, FIGS. 3D to 3G are schematic diagrams of various patient information cards corresponding to different patient information items according to an embodiment of the disclosure. In FIGS. 3D to 3G, the patient information cards 313a to 313m may, for example, respectively correspond to the patient information items 312a to 312m in FIG. 3B. In other words, when the patient information item 312a in FIG. 3B is selected, the item selection box 312 may display multiple patient information cards 313a corresponding to the patient information item 312a; when the patient information item 312b in FIG. 3B is selected, the item selection box 312 may display multiple patient information cards 313b corresponding to the patient information item 312b; when the patient information item 312d in FIG. 3B is selected, the item selection box 312 may display multiple patient information cards 313d corresponding to the patient information item 312d, but the disclosure is not limited thereto. When other patient information items are selected, the content displayed by the item selection box 312 should be deduced according to the aforementioned teaching, and details thereof are not repeated.

To facilitate further understanding, it is assumed that the user selects the patient information item 312a as the first patient information item in FIG. 3B, but the disclosure is not limited thereto.

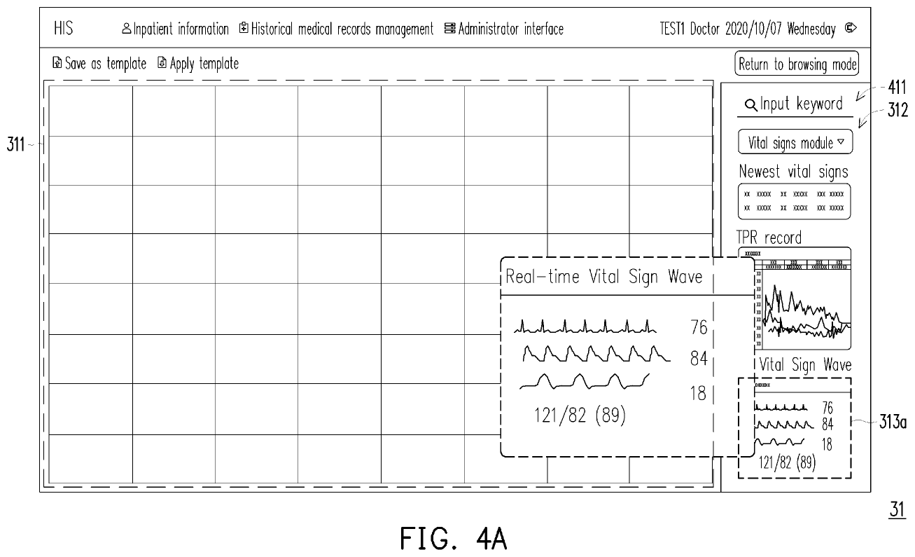

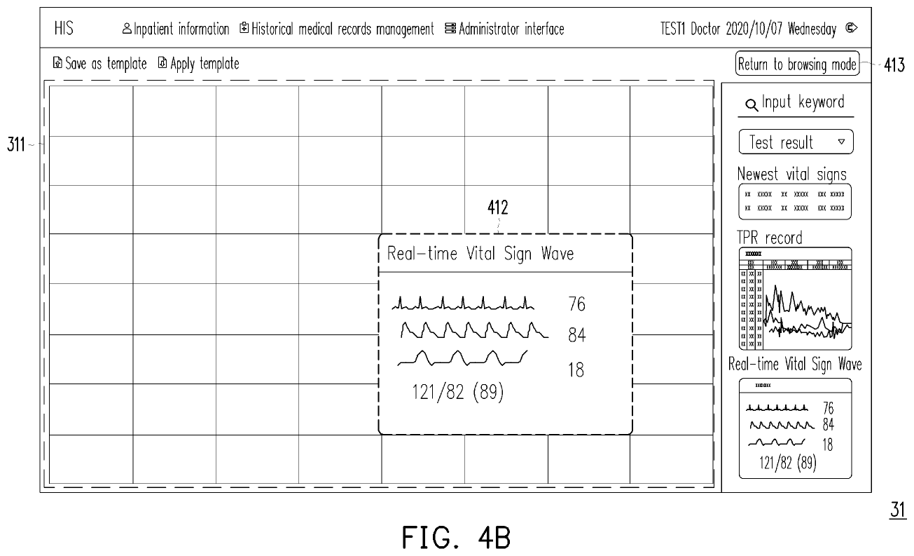

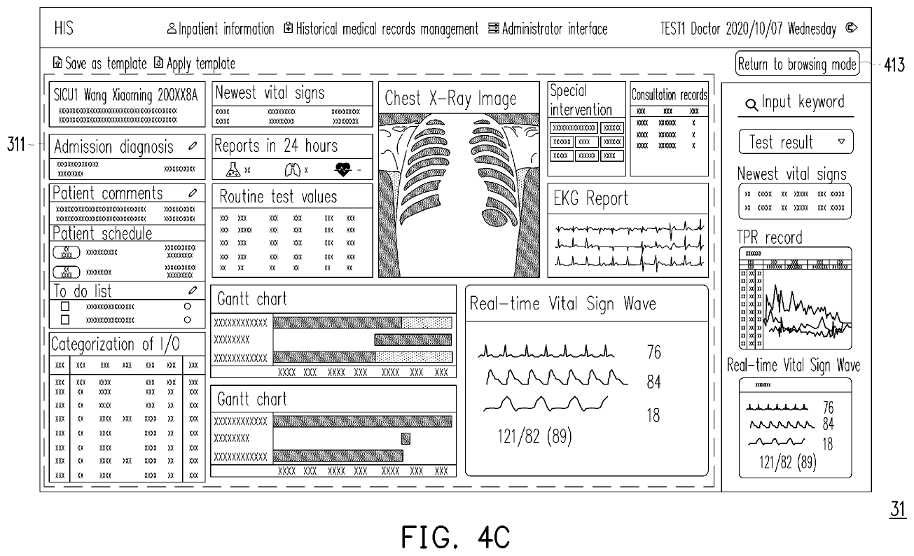

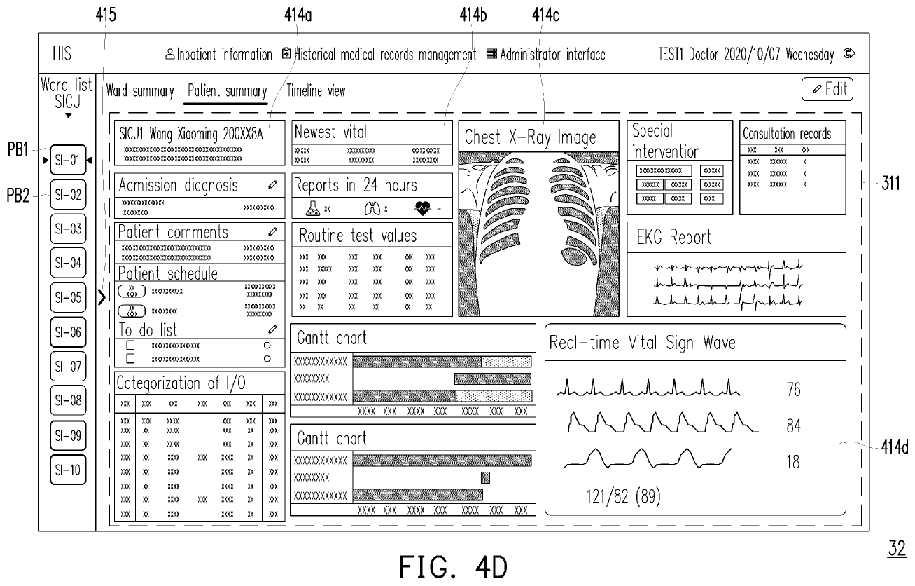

Referring to FIG. 4A, FIG. 4A is a schematic diagram showing multiple patient information cards according to an embodiment of the disclosure. In the embodiment, since the patient information item 312a is selected, the processor 104 may correspondingly display multiple corresponding patient information cards 313a for the user's reference.

In other embodiments, the user may also input a keyword of the patient information card to be searched in a search box 411 of the editing interface 31, and the item selection box 312 may display one or multiple patient information cards matching the keyword for the user's reference, but the disclosure is not limited thereto.

In FIG. 4A, the user may drag the required patient information card to a desired position on the first wallpaper 311 (which is referred to as a first position hereinafter) for placement. For example, the user may drag the patient information card 313a (which may be used to present information such as a patient's heart rate, a blood pressure, a body temperature, etc.) in FIG. 4A toward the desired first position.

Correspondingly, in step S230, in response to determining that the first patient information card (i.e. the patient information card 313a) corresponding to the first patient information item (i.e. the patient information item 312a) is dragged, the processor 104 may present the first patient information card in a corresponding first predetermined size during a dragging process of the first patient information card.

In the embodiment of the disclosure, different patient information cards may have the same or different predetermined sizes, i.e., a predetermined occupation space/area on the first wallpaper 311. In different embodiments, the predetermined size of each patient information card may be represented by corresponding n₁×m₁ grids, where n₁<N, m₁<M.

For example, the first predetermined size of the patient information card 313a in FIG. 4A is, for example, 4×3 grids. In this case, the processor 104 may present the patient information card 313a in a corresponding first predetermined size (i.e., 4×3 grids) during the dragging process of the patient information card 313a, but the disclosure is not limited thereto.

Referring to FIG. 4B, FIG. 4B is a schematic diagram of pinning the patient information card according to FIG. 4A. In FIG. 4B, it is assumed that the user drags the patient information card 313a to a first position 412 shown in FIG. 4B for placement. Correspondingly, in step S240, in response to determining that the first patient information card (i.e., the patient information card 313a) is dragged to and placed at the first position 412 on the first wallpaper 311, the processor 104 may pin the first patient information card to the first position 412 on the first wallpaper 311 in the corresponding first predetermined size (i.e., 4×3 grids).

In an embodiment, before the processor 104 pins the first patient information card to the first position 412 on the first wallpaper 311 in the corresponding first predetermined size (i.e., 4×3 grids), the processor 104 may first determine whether the first position 412 is pinned with other patient information cards, and whether the first patient information card being dragged exceeds the first wallpaper 311. In response to determining that the first position 412 is blank (that is, no patient information card is pinned), and the first patient information card being dragged does not exceed the first wallpaper 311, the processor 104 may then pin the first patient information card to the first position 412 on the first wallpaper 311 in the corresponding first predetermined size (i.e., 4×3 grids), but the disclosure is not limited thereto.

In other embodiments, steps S220 to S240 may be repeatedly executed until the user has placed each patient information card he needs on the first wallpaper 311, but the disclosure is not limited thereto.

Referring to FIG. 4C, FIG. 4C is a schematic diagram of finishing editing the first wallpaper according to FIG. 4B. In the embodiment, it is assumed that the user has placed each selected first patient information card to the corresponding first position on the first wallpaper 311 according to his needs, and the first wallpaper 311 in FIG. 4C is formed. In this case, the user may click/trigger an end editing button 413 in the editing interface 31.

In an embodiment, the content of each patient information card in FIG. 4C may be only a temporary thumbnail without any actual patient information, but the disclosure is not limited thereto.

Correspondingly, in step S250, in response to determining that the end editing button 413 in the editing interface 31 is triggered, the processor 104 may switch the user interface 30 to a browsing interface 32, and present the first wallpaper 311 in the browsing interface, where the first wallpaper 311 may include the corresponding first patient information card in the first position, and the first patient information card presents information corresponding to the current patient of interest.

Referring to FIG. 4D, FIG. 4D is a schematic diagram of the browsing interface according to FIG. 4C. In the embodiment, it is assumed that the current patient of interest of the user is a first patient named “Wang Xiaoming”, each patient information card on the first wallpaper 311 in the browsing interface 32 presents various patient information corresponding to this first patient. For example, a patient information card 414a may present identity information of the first patient; a patient information card 414b may present a heart rate, a blood pressure, a body temperature and other values of the first patient; a patient information card 414c may present an X-ray photo of the first patient; and a patient information card 414d may present an electrocardiogram of the first patient, but the disclosure is not limited thereto.

In different embodiments, the content of the patient information cards 414b and 414d may be synchronously obtained by the processor 104 from related measuring devices of the first patient through a related data gateway, but the disclosure is not limited thereto. In addition, the processor 104 may obtain the X-ray photo of the first patient (which may be converted into a jpg file by PACS) through a related digital image storage communication system (PACS) gateway or database, and present it in the patient information card 414c, but the disclosure is not limited thereto.

In some embodiments, regarding the patient information card that provides medical data, the user may customize a normal range of related data, and when the processor 104 determines that certain medical data exceeds the corresponding normal range, the processor 104 may adjust the related patient information card to a specific appearance, color or present a special warning symbol to remind the user, but the disclosure is not limited thereto.

In other embodiments, when the user clicks on another patient button corresponding to another patient in the browsing interface 32, for example, clicks the patient button PB2, the processor 104 may switch the current patient of interest to the another patient, and correspondingly adjust the content of each patient information card on the first wallpaper 311 to correspond to the another patient. In this case, each patient information card on the first wallpaper 311 in the browsing interface 32 may present various patient information corresponding to the another patient. For example, the patient information card 414a may present identity information of the another patient; the patient information card 414b may present a heart rate, a blood pressure, a body temperature and other values of the another patient; the patient information card 414c may present an X-ray photo of the another patient; and the patient information card 414d may present an electrocardiogram of the another patient, but the disclosure is not limited thereto.

In an embodiment, the patient button corresponding to the patient may be marked with a specific button color and a warning indicator. For example, a first patient button and a second patient button respectively corresponding to the first patient and the another patient may be respectively marked with a first warning indicator and a second warning indicator, where the first warning indicator and the second warning indicator may be changed in real-time according to a degree of urgency of the first patient and the another patient.

For example, the first warning indicator and the second warning indicator may be respectively implemented as outer frames of the first patient button and the second patient button. In this case, a color of the outer frame of the first patient button may be changed in real-time according to the urgency degree of the first patient. For example, when some real-time physiological data of the first patient exceeds a predetermined normal range, the processor 104 may adjust the outer frame of the first patient button to red, so that the user may take corresponding medical measures in time. In addition, when the real-time physiological data of the first patient is within the normal range, the processor 104 may adjust the outer frame of the first patient button to green (or do not present the outer frame), so that the user knows that the first patient has no emergency situation at present. Similarly, the processor 104 may perform the above adjustment on the second patient button, but the disclosure is not limited thereto.

In some embodiments, the first patient button and the second patient button may also be respectively marked with a first button color and a second button color, and the first button color and the second button color respectively correspond to a degree of required care of the first patient and the another patient. For example, when the condition of the first patient is more severe and needs more frequent confirmations, the processor 104 may, for example, set the first button color to red to let the user know that the first patient needs more attention. On the other hand, when the condition of the first patient is less severe and does not require frequent confirmation, the processor 104 may, for example, set the first button color to green to let the user know that the first patient does not need more attention, but the disclosure is not limited thereto.

It may be seen from the above that the embodiment of the disclosure allows the user to adjust the content, size and layout of each patient information card in the browsing interface according to actual needs, so that the user may view various information of the patients in his own way.

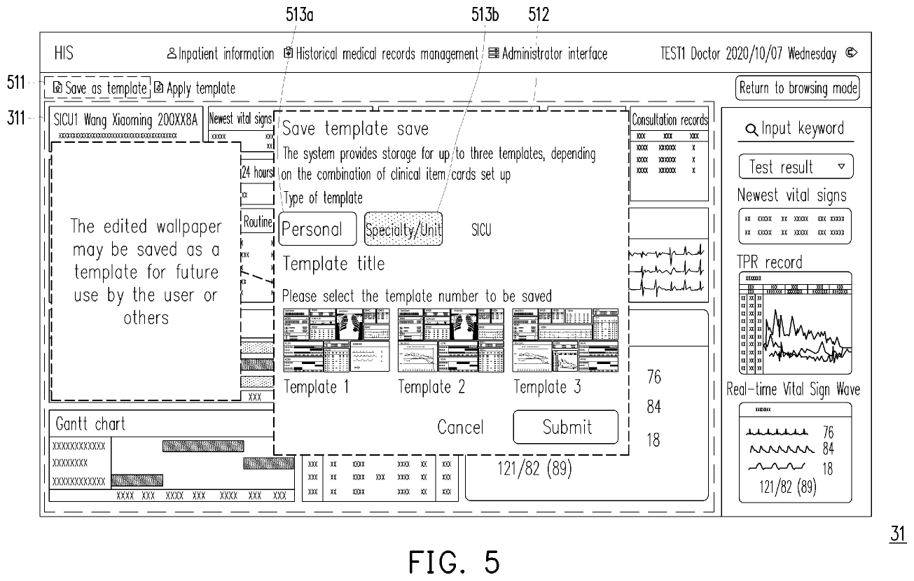

In some embodiments, after the user finishes editing the first wallpaper, the user may save it as a template for future use by himself or others. Referring to FIG. 5, FIG. 5 is a schematic diagram of a saved template according to FIG. 4C.

In FIG. 5, after the user finishes editing the first wallpaper 311, the user may click/trigger a save as template button 511 in the editing interface 31. Correspondingly, in response to determining that the save as template button 511 in the editing interface 31 is triggered, the processor 104 may provide a save template dialog box 512, where the save template dialog box 512 may include a for personal use template button 513a and a for public use by unit template button 513b.

In the situation of FIG. 5, after the user finishes editing the first wallpaper 311, if the user only wants to use the layout presented by the first wallpaper 311 for personal use, the user may click/trigger the for personal use template button 513a, and click submit. In this case, in response to determining that the for personal use template button 513a is triggered, the processor 104 may save a current layout of the first wallpaper 311 as a for personal use template exclusively to/corresponding to an account for direct application of the user in the future.

On the other hand, if the user only wants to share the layout presented by the first wallpaper 311 with other colleagues belonging to a certain working unit (for example, the shown SICU), the user may click/trigger the for public use by unit template button 513b, and click submit. In this case, in response to determining that the for public use by unit template button 513b is triggered, the processor 104 may save the current layout of the first wallpaper 311 as a for public use by unit template that is shared by the above-mentioned working unit for the use of other colleagues of the above-mentioned working unit, but the disclosure is not limited thereto.

In some embodiments, when the user edits the first wallpaper 311, the user may also directly apply other existing templates. Details thereof are provided as follows.

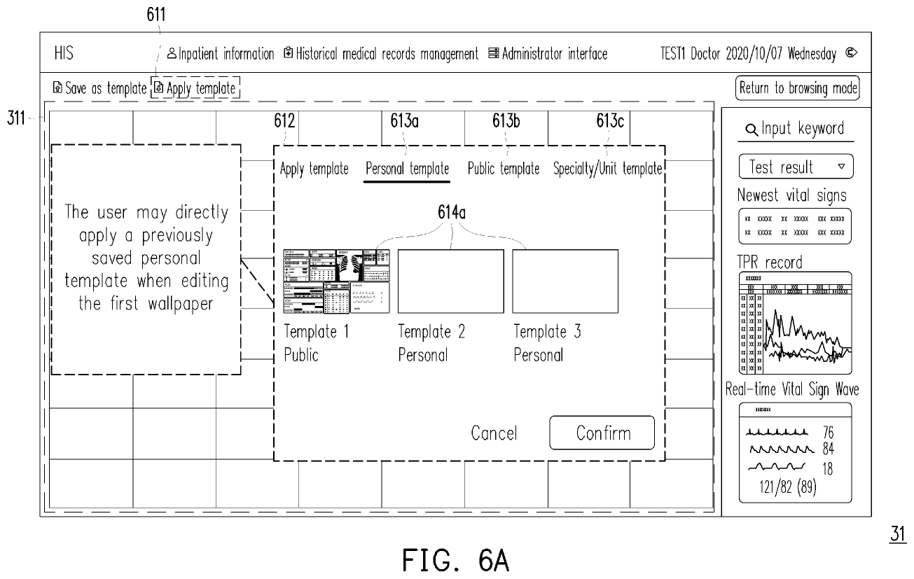

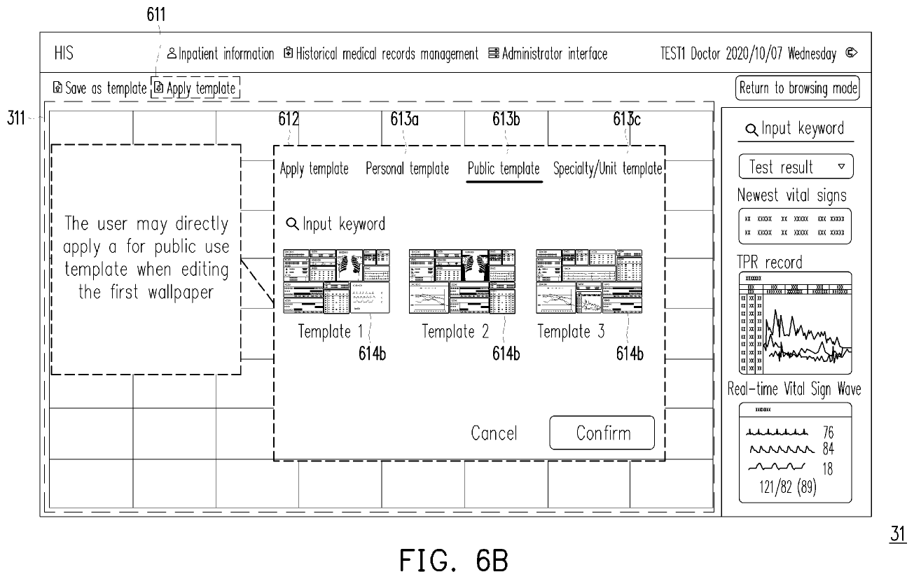

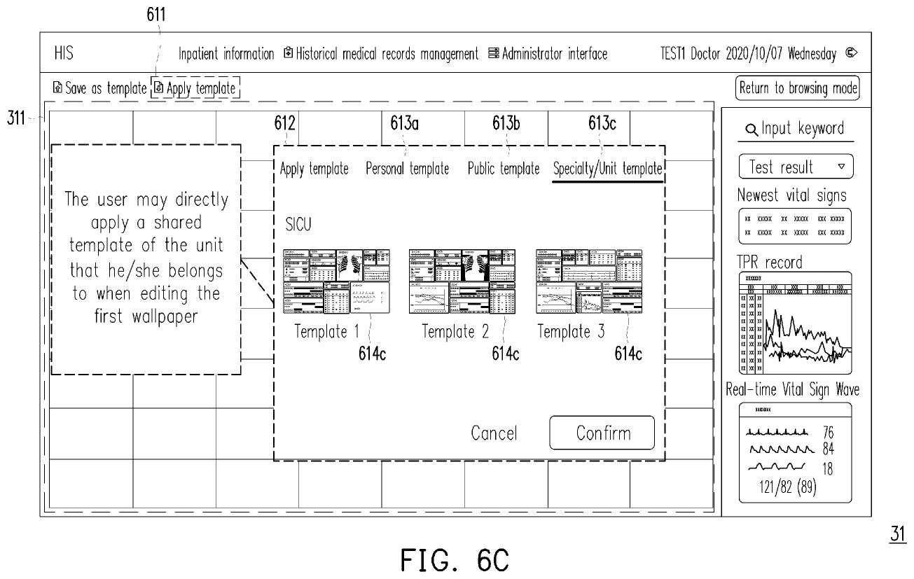

Referring to FIG. 6A, FIG. 6A is a schematic diagram of applying a for personal use template according to an embodiment of the disclosure. In the embodiment, after entering the editing interface 31, it is assumed that the user wants to directly apply other existing templates as the layout of the first wallpaper 311, the user may click/trigger an applying template button 611 in the editing interface 31.

In this case, in response to determining that the applying template button 611 is triggered, the processor 104 may provide a template application dialog box 612, where the template application dialog box 612 may include a for personal use template button 613a, a for public use template button 613b, and a for public use by unit template button 613c.

In FIG. 6A, when the user wants to apply his previously saved for personal use template, the user may click/trigger the for personal use template button 613a. In response to determining that the for personal use template button 613a is triggered, the processor 104 may provide a for personal use template 614a exclusively to the current user of the electronic device 100 in the template application dialog box 612.

On the other hand, when the user wants to apply a for public use template that may be accessed by all users, the user may click/trigger the for public use template button 613b. Referring to FIG. 6B, FIG. 6B is a schematic diagram of applying the public template according to FIG. 6A. In the embodiment, in response to determining that the for public use template button 613b is triggered, the processor 104 may provide the public template 614b in the template application dialog box 612.

In another embodiment, when the user wants to apply a for public use by unit template that is shared by a certain working unit, the user may click/trigger the for public use by unit template button 613c. Referring to FIG. 6C, FIG. 6C is a schematic diagram of applying the for public use by unit template according to FIG. 6A. In the embodiment, in response to determining that the for public use by unit template button 613c is triggered, the processor 104 may provide a for public use by unit template 614c that is shared by a certain working unit (for example, the SICU) in the template application dialog box 612.

In an embodiment, in response to determining that a specific template in the for personal use template 614a, the public template 614b, and the for public use by unit template 614c is selected, and the user clicks a confirmation button shown in FIGS. 6A to 6C, the processor 104 may switch the user interface 30 to the browsing interface, and present a specific wallpaper corresponding to the above-mentioned specific template in the browsing interface, where the specific wallpaper may include specific patient information cards, and each specific patient information card presents specific patient information corresponding to the current patient of interest.

For example, it is assumed that the specific template selected by the user has the same layout as the first wallpaper in FIG. 4D, when the processor 104 switches the user interface 30 to the browsing interface, the content of the browsing interface will be the same as that shown in FIG. 4D, but the disclosure is not limited thereto.

In some embodiments, the user may also add other wallpapers (which are also referred to as paging) in the editing interface 31 according to actual needs, and the user may access the information of these wallpapers through corresponding paging buttons in the browsing interface.

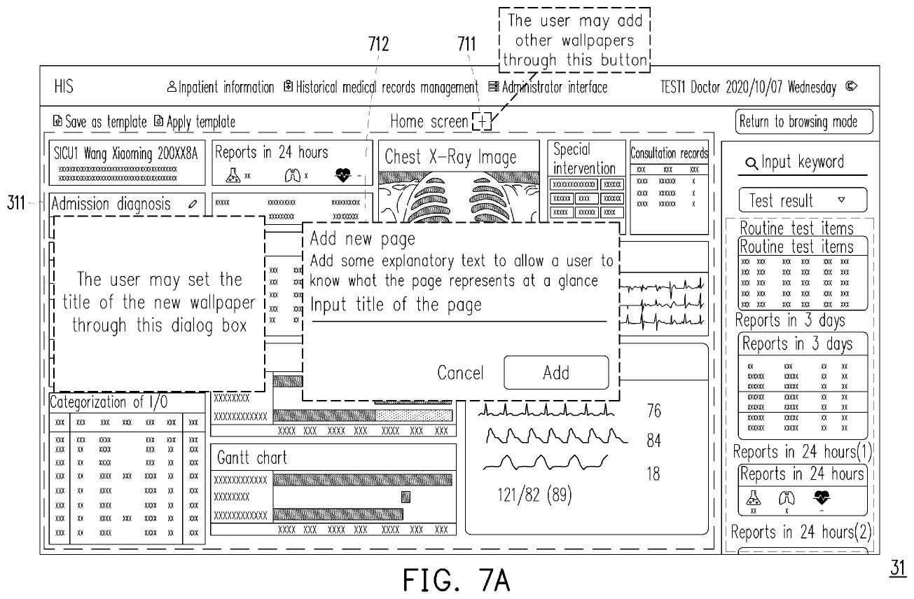

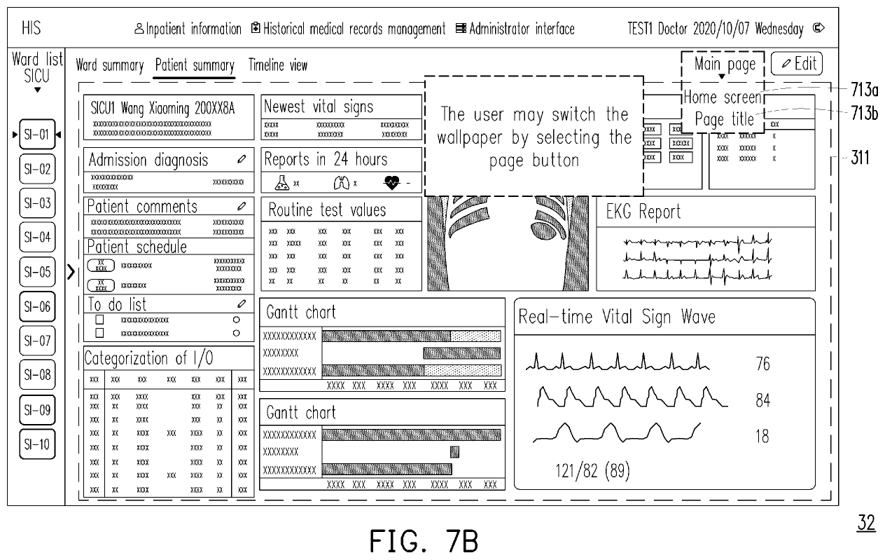

Referring to FIG. 7A, FIG. 7A is a schematic diagram of a newly added page according to an embodiment of the disclosure. In the embodiment, when the user regards that a space of the first wallpaper 311 may no longer fit other patient information cards, the user may, for example, click/trigger a add new page button 711 in the editing interface 31 to try to edit a new wallpaper (which is referred to as a second wallpaper hereinafter). Correspondingly, the processor 104 may, for example, provide a add new page dialog box 712 for the user to input information such as a title of the second wallpaper, but the disclosure is not limited thereto.

In an embodiment, after the user confirms the title of the second wallpaper, the processor 104 may provide the second wallpaper and the item selection box 312 in the editing interface 31, and the content of the editing interface 31 will be the same or similar to that shown in FIG. 3B, but the disclosure is not limited thereto.

Thereafter, the user may add the required patient information cards to the second wallpaper 311 according to the same way of editing the first wallpaper 311. In overall, in response to determining that the second patient information item among the patient information items 312a to 312m is selected, the processor 104 may display multiple patient information cards corresponding to the second patient information item in the item selection box 312. In response to determining that the second patient information card in the patient information cards corresponding to the second patient information item is dragged, the processor 104 may present the second patient information card in a corresponding second predetermined size during the process of dragging the second patient information card. In response to determining that the second patient information card is dragged to a second position on the second wallpaper for placement, the processor 104 may pin the second patient information card to the second position on the second wallpaper in the corresponding second predetermined size. Details of the above steps may be deduced by referring to descriptions of the above embodiments, which will not be repeated.

Referring to FIG. 7B, FIG. 7B is a schematic diagram of switching pages according to FIG. 7A. In the embodiment, it is assumed that the user has finished editing the first wallpaper 311 and the second wallpaper, and the processor 104 has switched the user interface 30 to the browsing interface 32.

As shown in FIG. 7B, the browsing interface 32 may include a first page button 713a and a second page button 713b respectively corresponding to the first wallpaper 311 and the second wallpaper. In FIG. 7B, it is assumed that the user currently selects the first page button 713a, and the browsing interface 32 may correspondingly display the first wallpaper 311.

In an embodiment, when the user wants to view the patient information cards on the second wallpaper, the user may click/trigger the second page button 713b. In response to determining that the second page button 713b is triggered, the processor 104 may present the second wallpaper in the browsing interface 32, where the second wallpaper may include each of the patient information cards previously edited by the user, and each patient information card presents the patient information corresponding to the current patient of interest, but the disclosure is not limited thereto.

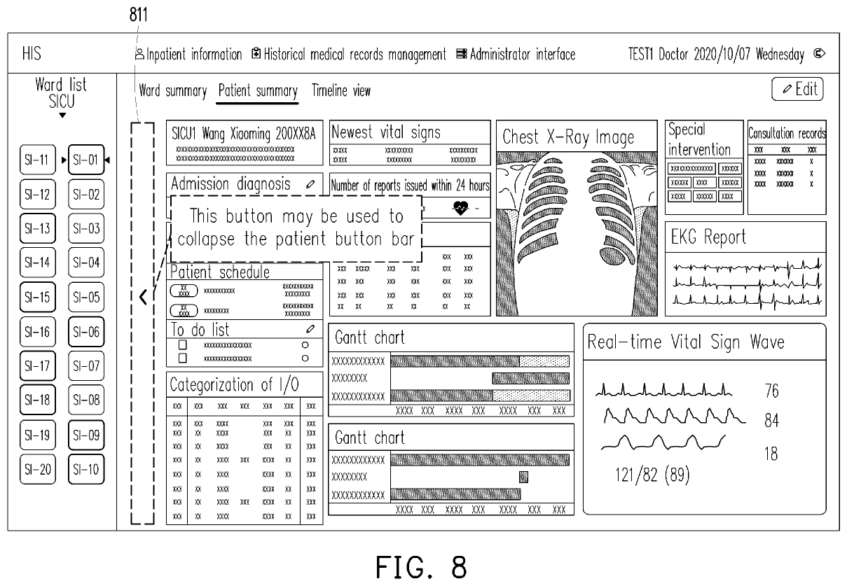

Referring to FIG. 8, FIG. 8 is a schematic diagram of expanding a patient button field according to FIG. 4D. In the embodiment, when the user clicks/triggers an expanding button 415 in FIG. 4D, the processor 104 may expand the patient button field as shown in FIG. 8 to allow the user to view the patient buttons of other patients belonging to a current ward of interest (such as SICU). Moreover, after expanding the patient button field, the processor 104 may switch the expanding button 415 in FIG. 4D to a retracting button 811 shown in FIG. 8.

When the user clicks/triggers the retracting button 811 in FIG. 8, the processor 104 may retract the patient button field to present the state of FIG. 4D, but the disclosure is not limited thereto.

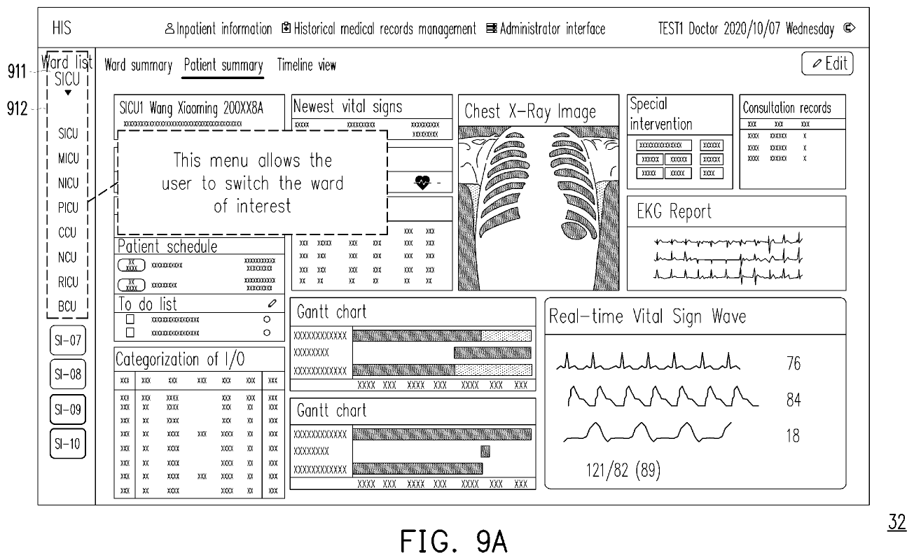

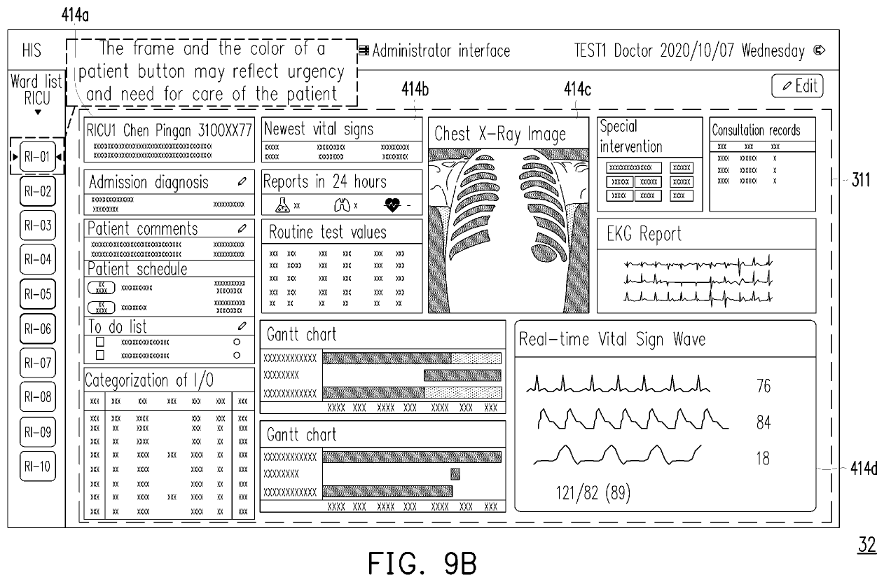

Referring to FIG. 9A, FIG. 9A is a schematic diagram of ward switching according to an embodiment of the disclosure. In FIG. 9A, the browsing interface 32 may include a ward menu button 911, which may be used by the user to switch the ward of interest.

In an embodiment, when the user clicks/triggers the ward menu button 911, the processor 104 may provide a ward menu 912, which may include ward buttons corresponding to various wards. It is assumed that when the user wants to pay attention to a patient belonging to a respiratory intensive care unit (RICU), the user may click the corresponding ward button to switch the ward.

Referring to FIG. 9B, FIG. 9B is a schematic diagram of another browsing interface according to FIG. 9A. In FIG. 9B, it is assumed that the processor 104 has switched the ward of interest to RICU, the first wallpaper 311 of the browsing interface 31 may be changed to display related patient information of another patient (which is referred to as a third patient hereinafter) belonging to RICU while maintaining the layout.

As shown in FIG. 9B, the patient information card 414a may present identity information of the third patient; the patient information card 414b may present a heart rate, a blood pressure, a body temperature and other values of the third patient; the patient information card 414c may present an X-ray photo of the third patient; and the patient information card 414d may present an electrocardiogram of the third patient, but the disclosure is not limited thereto.

In the embodiment, each patient button may also be designed with the previously described warning indicator (such as the outer frame) and button color, and details thereof are not repeated.

In an embodiment, different patient information cards belonging to the same patient information item may be used to present (a part of) the same patient information, but these patient information cards may have different predetermined sizes to allow the user to edit the wallpaper in the editing interface more flexibly.

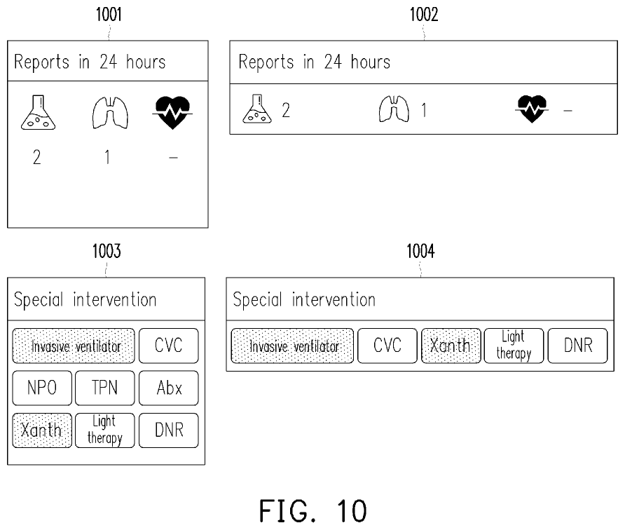

Referring to FIG. 10, FIG. 10 is a schematic diagram of patient information cards with different predetermined sizes according to an embodiment of the disclosure. In FIG. 10, patient information cards 1001 and 1002 may be used to present the same patient information, but the predetermined sizes of the patient information cards 1001 and 1002 may be respectively 2×1 and 1×2 for the user to choose according to actual needs.

In addition, the patient information presented by the patient information cards 1003 and 1004 are partially the same, and the predetermined sizes of the patient information cards 1003 and 1004 may be respectively 2×1 and 1×2 for the user to choose according to actual needs.

In some embodiments, the patient information cards of different sizes may have different clinical review effects and meanings, and the user may flexibly select the sizes and contents of the required patient information cards according to review requirements. For example, regarding a small-sized patient information card belonging to an examination report, it may, for example, only provide a number of examination items that have been reported within 24 hours. On the other hand, regarding the medium and large-sized patient information cards belonging to the examination report, it may, for example, provide the user with access to an examination value trend result of special examination items, but the disclosure is not limited thereto.

In some embodiments, the processor 104 further adjusts the size of the patient information card on the wallpaper in response to a drag operation of the user on the outer frame of the patient information card that has been pinned to the wallpaper. In one embodiment, in response to the size change, the processor 104 may also adjust a display content of the patient information card accordingly.

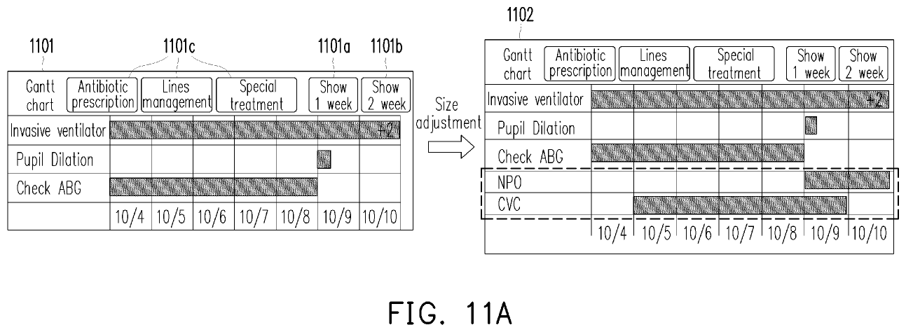

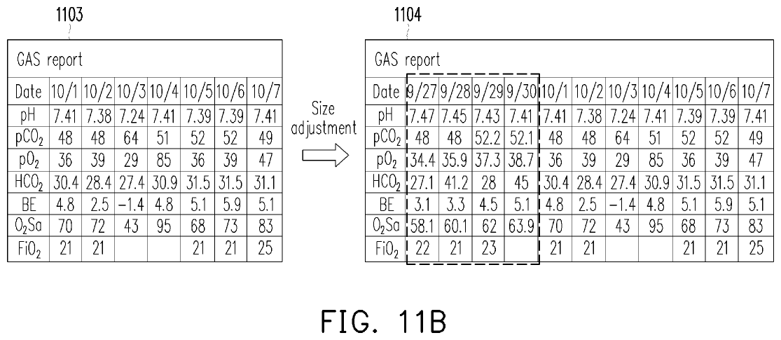

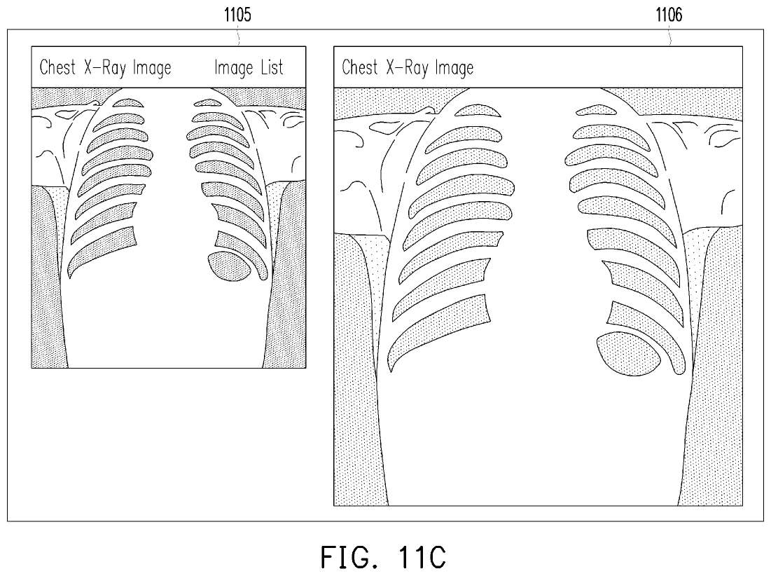

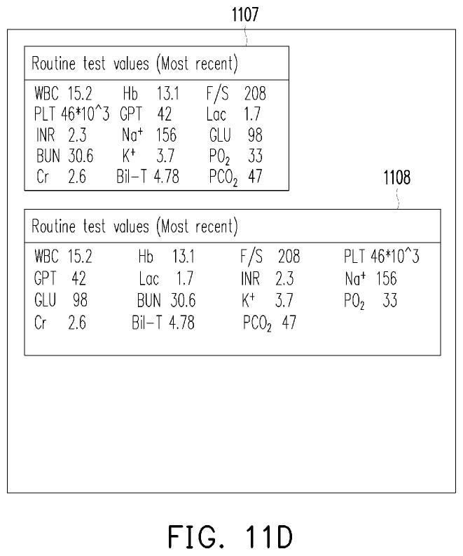

Referring to FIGS. 11A to 11D, FIGS. 11A to 11D are schematic diagrams of adjusting sizes of patient information cards according to different embodiments of the disclosure. In FIG. 11A, a patient information card 1101 is, for example, a Gantt chart card, which may be used to, for example, provide one or more medical information Gantt charts of the patient. In FIG. 11A, the above medical information Gantt chart may be used to present specific medical information of the patient in a specific time interval, such as a time interval when a certain medical device (such as an invasive ventilator) has been installed, a time interval of performing a certain clinical test (such as a pupil dilation test), and a time interval of performing a certain medical treatment, etc., but the disclosure is not limited thereto.

In an embodiment, a color of each medical information Gantt chart in the patient information card 1101 may correspond to a degree of abnormality of the specific medical information. For example, when a use time of the invasive ventilator of the patient has exceeded a normal time range, the processor 104 may display the corresponding medical information Gantt chart in red, but the disclosure is not limited thereto. In addition, the medical information Gantt chart may also indicate a length of time overdue. For example, “+2” in FIG. 11A may indicate that a time of using the invasive ventilator has expired by 2 days, but the disclosure is not limited thereto.

In FIG. 11A, the patient information card 1101 may have a first time scale button 1101a and a second time scale button 1101b, which may respectively correspond to a first specified time interval and the second specified time interval. In an embodiment, when the first time scale button 1101a is selected, the processor 104 may adjust the specified time interval used in the medical information Gantt chart to the first specified time interval (for example, within a week). In addition, when the second time scale button 1101b is selected, the processor 104 may adjust the specified time interval used in the medical information Gantt chart to the second specified time interval (for example, within two weeks), but the disclosure is not limited thereto.

Moreover, the patient information card 1101 may further have Gantt chart buttons 1101c for the user to switch the medical information Gantt chart to be concerned, but the disclosure is not limited thereto.

In FIG. 11A, after the user pins the patient information card 1101 on the wallpaper, the user may expand the patient information card 1101 into a patient information card 1102 by dragging the outer frame of the patient information card 1101 longitudinally. It may be seen from FIG. 11A that a longitudinal roller in the patient information card 1101 may disappear after the size adjustment, but the disclosure is not limited thereto.

In FIG. 11B, a patient information card 1103 may include multiple data corresponding to multiple time points. In the embodiment, after the user pins the patient information card 1103 to the wallpaper, the user may expand the patient information card 1103 into a patient information card 1104 by dragging the outer frame of the patient information card 1103 laterally.

In FIG. 11B, in response to determining that the size of the patient information card 1103 is expanded into the patient information card 1104 by the drag operation, the processor 104 may present more data corresponding to more time points in the patient information card 1104, but the disclosure is not limited thereto.

In FIG. 11C, a patient information card 1105 may be used to, for example, present a medical image, and when the user tries to expand the patient information card 1105 (for example, the editing interface receives a drag operation of pulling the outer frame of the patient information card 1105 outward through a cursor or a touch screen), the processor 104 may, for example, expand the patient information card 1105 (with a size of, for example, 2×2) into a patient information card 1106 (with a size of, for example, 3×3) in a proportional manner, but the disclosure is not limited thereto.

In FIG. 11D, a patient information card 1107 may be used to present patient information with a fixed amount of data. In this case, when the user tries to enlarge the patient information card 1107 (a size thereof is, for example, 2×2) (for example, the editing interface receives a drag operation of pulling the outer frame of the patient information card 1107 outward through a cursor or a touch screen), the processor 104 may, for example, arbitrarily expand the size of the patient information card 1107 according to the user needs. The expanded patient information card 1107 is, for example, a patient information card 1108 (a size thereof is, for example, 2×3), but the disclosure is not limited thereto.

In some embodiments, regarding certain patient information cards of a warning type, related decision-making assistance prompt messages may also be designed for the user's reference.

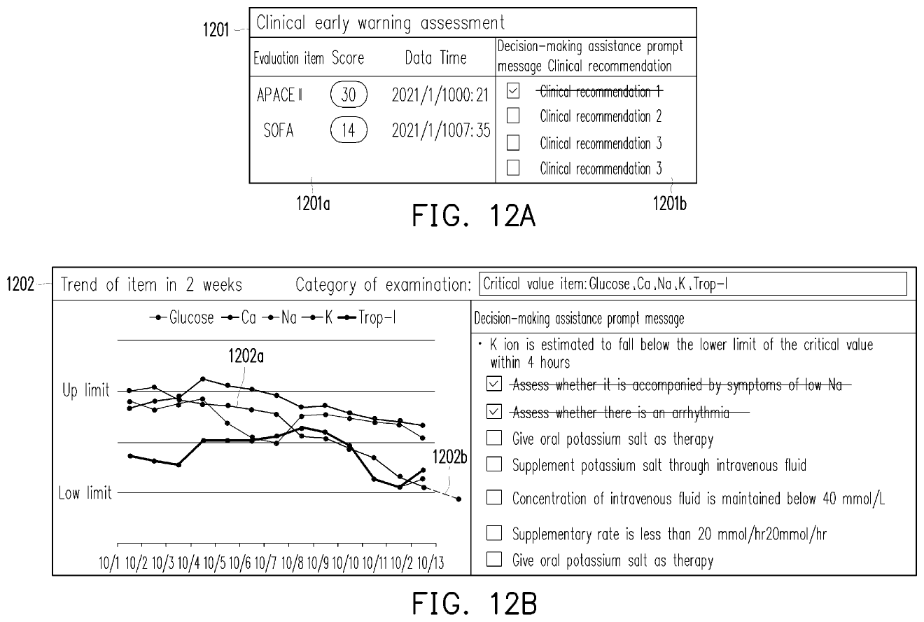

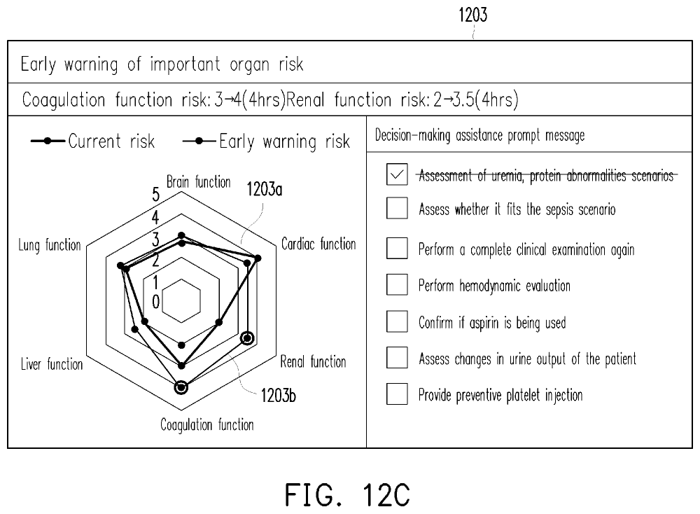

Referring to FIGS. 12A to 12C, FIGS. 12A to 12C are schematic diagrams of patient information cards of a warning type according to different embodiments of the disclosure. In FIG. 12A, a patient information card 1201 may include fields 1201a and 1201b. The field 1201a may be used to present related patient information, and the field 1201b may be used to present related decision-making assistance prompt messages (such as various clinical recommendations), but the disclosure is not limited thereto.

In FIG. 12B, a patient information card 1202 may be used to present an examination item trend graph, where the examination item trend graph includes a historical variation trend and a predicted variation trend of a specific examination item. For example, the above-mentioned specific examination item is, for example, a potassium index of the patient, and the patient information card 1202 may include a historical variation trend 1202a and a predicted variation trend 1202b of the potassium index. In different embodiments, the processor 104 may predict data points in the predicted variation trend 1202b based on the data points in the historical variation trend 1202a according to a related prediction algorithm, and the above prediction algorithm may include but is not limited to linear model, random forest, XGBoost, support vector regression (SVR), LASSO, Ensemble, etc.

In addition, the patient information card 1202 may also include a field for presenting a related decision-making assistance prompt message, but the disclosure is not limited thereto.

In FIG. 12C, a patient information card 1203 may be used to present an early warning map of organ risk of the patient, where the early warning map of the organ risk may be in form of a radar map, and the early warning map of the organ risk may indicate a current risk score and a predicted risk score of each of multiple organ functions. In FIG. 12C, the considered plurality of organ functions are a brain function, a lung function, a liver function, a blood coagulation function, a cardiovascular function, and a renal function, and the corresponding current risk scores thereof may be determined based on corresponding sequential organ failure assessment scores (SOFA scores), but the disclosure is not limited thereto.

As shown in FIG. 12C, the current risk scores and predicted risk scores corresponding to the above organ functions may be respectively as radar maps 1203a and 1203b, where the predicted risk score of each organ function may also be determined by the processor 104 based on the above-mentioned prediction algorithm, but the disclosure is not limited thereto.

In some embodiments, in response to determining that the predicted risk score of the specific organ function of the aforementioned organ functions exceeds a predetermined threshold, the processor 104 may also provide a warning prompt corresponding to the specific organ function. In FIG. 12C, it is assumed that the predicted risk scores of each of the blood coagulation function and the renal function exceed the corresponding predetermined thresholds, the processor 104 may circle the predicted risk scores of each of the blood coagulation function and the renal function in the shown circles to serve as a warning prompt, but the disclosure is not limited thereto.

In summary, the disclosure allows the user to configure the content, size and layout of each patient information card in the browsing interface according to his needs, so that the user may view various patient information in his own way.

Moreover, after the user completes editing the wallpaper, the user may save it as a for personal use template or a for public use by unit template for direct application by himself or others in the future. In addition, the user may also directly apply an existing template (such as a for personal use template, a for public use by unit template, and a for public use template) to reduce a workload of wallpaper editing. Moreover, the user may also configure a layout of the patient information cards in different wallpapers more flexibly by adding pages.

In addition, the browsing interface may also be provided with patient buttons corresponding to different patients, and each patient button may be designed with a color and a warning indicator corresponding to the patient's condition for the user's reference.

It will be apparent to those skilled in the art that various modifications and variations can be made to the disclosed embodiments without departing from the scope or spirit of the disclosure. In view of the foregoing, it is intended that the disclosure covers modifications and variations provided they fall within the scope of the following claims and their equivalents.

What is claimed is:

1. A patient information presentation method, adapted to an electronic device, and comprising:
switching a user interface to an editing interface in response to determining that an edit button in the user interface is triggered, wherein the editing interface comprises a first wallpaper and a plurality of patient information cards;
pinning a first patient information card to a first position on the first wallpaper in a corresponding first predetermined size in response to determining that the first patient information card in the plurality of patient information cards is dragged to and placed at the first position on the first wallpaper; and
switching the user interface to a browsing interface in response to determining that an end editing button in the editing interface is triggered, and presenting the first wallpaper in the browsing interface, wherein the first wallpaper comprises the first patient information card set in the first position, and the first patient information card presents information corresponding to a current patient of interest.

2. The patient information presentation method as claimed in claim 1, wherein
after the step of pinning the first patient information card to the first position on the first wallpaper in the corresponding first predetermined size, the method further comprising:
providing a save template dialog box in response to determining that a save as template button in the editing interface is triggered, wherein the save template dialog box comprises a for personal use template button and a for public use by unit template button;
saving a current layout of the first wallpaper as a for personal use template exclusively to an account in response to determining that the for personal use template button is triggered; and
saving the current layout of the first wallpaper as a for public use by unit template that is shared by a working unit in response to determining that the for public use by unit template button is triggered.

3. The patient information presentation method as claimed in claim 1, wherein the editing interface further comprises an applying template button, the method further comprising:
providing a template application dialog box in response to determining that the applying template button is triggered, wherein the template application dialog box comprises a for personal use template button, a for public use template button, and a for public use by unit template button;
providing at least one for personal use template corresponding to an account in the template application dialog box in response to determining that the for personal use template button is triggered;
providing at least one for public use template in the template application dialog box in response to determining that the for public use template button is triggered;
providing at least one for public use by unit template that is shared by a working unit in the template application dialog box in response to determining that the for public use by unit template button is triggered; and
presenting a specific wallpaper corresponding to a specific template in the browsing interface in response to determining that the specific template among the at least one for personal use template, the at least one for public use template, and the at least one for public use by unit template is selected, wherein the specific wallpaper comprises at least one specific patient information card, and each of the specific patient information cards presents at least one specific patient information corresponding to the current patient of interest.

4. The patient information presentation method as claimed in claim 1, wherein the editing interface further comprises an item selection box, the item selection box comprises a plurality of patient information items, wherein the plurality of patient information items comprise at least one of vitals sign information, diagnosis and annotation information, examination result information, imaging examination information, intake/output record information, medication record information, doctor order information, vascular lines record information, consultation record information, and ventilator record information, and the plurality of patient information items comprise a first patient information item corresponding to the plurality of patient information items, and the method further comprising:
displaying the plurality of patient information cards corresponding to the first patient information item in the item selection box in response to determining that the first patient information item is clicked,
wherein the plurality of patient information cards further comprise a second patient information card corresponding to a second predetermined size, and patient information corresponding to the second patient information card and the first patient information card are partially the same.

5. The patient information presentation method as claimed in claim 4, wherein the editing interface further comprises a add new page button, and the method further comprises:
providing a second wallpaper and the item selection box in the editing interface in response to determining that the add new page button in the editing interface is triggered;
displaying a plurality of patient information cards corresponding to a second patient information item in the item selection box in response to determining that the second patient information item in the plurality of patient information items is clicked;
presenting a third patient information card in a corresponding third predetermined size in a process of dragging the third patient information card in response to determining that the third patient information card in the plurality of patient information cards corresponding to the second patient information item is dragged; and
pinning the third patient information card to a second position on the second wallpaper in the corresponding third predetermined size in response to determining that the third patient information card is dragged to and placed at the second position on the second wallpaper.

6. The patient information presentation method as claimed in claim 5, wherein the browsing interface comprises a first page button and a second page button respectively corresponding to the first wallpaper and the second wallpaper, and the method further comprising:
presenting the second wallpaper in the browsing interface in response to determining that the second page button is triggered, wherein the second wallpaper comprises the third patient information card disposed at the second position, and the third patient information card presents another information corresponding to the current patient of interest.

7. The patient information presentation method as claimed in claim 1, further comprising presenting the first patient information card in the corresponding first predetermined size during the process of dragging the first patient information card, wherein the first wallpaper comprises N×M grids, and the first predetermined size is n₁×m₁ grids, where N, M, n₁, m₁ are positive integers, and n₁<N, m₁<M.

8. The patient information presentation method as claimed in claim 1, wherein after the step of pinning the first patient information card to the first position on the first wallpaper in the corresponding first predetermined size, the method further comprising:
adjusting a size of the first patient information card on the first wallpaper in response to the editing interface receiving a drag operation on an outer frame of the first patient information card.

9. The patient information presentation method as claimed in claim 8, wherein in response to determining that the information of the current patient of interest comprises a plurality of data corresponding to a plurality of time points, the method further comprising:
presenting more data corresponding to more time points in the first patient information card in response to determining that the size of the first patient information card is expanded by the drag operation.

10. The patient information presentation method as claimed in claim 1, wherein the current patient of interest is a first patient belonging to a first ward, and the browsing interface further comprises a first patient button and a second patient button respectively corresponding to the first patient and another patient;
switching the current patient of interest to the another patient in response to determining that the second patient button is triggered, and displaying the first wallpaper in the browsing interface, wherein the first wallpaper comprises the first patient information card set in the first position, and the first patient information card presents the information corresponding to the current patient of interest.

11. The patient information presentation method as claimed in claim 10, wherein the first patient button and the second patient button are respectively marked with a first warning indicator and a second warning indicator, wherein the first warning indicator and the second warning indicator are changed in real-time according to a degree of urgency of the first patient and the another patient,
wherein the first patient button and the second patient button are respectively marked with a first button color and a second button color, and the first button color and the second button color respectively correspond to a degree of required care of the first patient and the another patient.

12. The patient information presentation method as claimed in claim 1, wherein the current patient of interest is a first patient belonging to a first ward, the browsing interface further comprises a ward button corresponding to a second ward, and the method further comprising:
presenting a patient button corresponding to the second ward in the browsing interface in response to determining that the ward button is triggered; and
switching the current patient of interest to a patient corresponding to the patient button of the second ward in response to determining that the patient button corresponding to the second ward is triggered, and presenting the first wallpaper in the browsing interface, wherein the first wallpaper comprises the first patient information card set in the first position, and the first patient information card presents the information corresponding to the current patient of interest.

13. The patient information presentation method as claimed in claim 1, wherein the first patient information card comprises a Gantt chart card, the Gantt chart card comprises a medical information Gantt chart corresponding to the current patient of interest, wherein the medical information Gantt chart presents a specific medical information of the current patient of interest in a first specified time interval.

14. The patient information presentation method as claimed in claim 13, wherein a color of the medical information Gantt chart corresponds to a degree of abnormality of the specific medical information, and the medical information Gantt chart indicates a length of time overdue.

15. The patient information presentation method as claimed in claim 13, wherein the medical information Gantt chart comprises a first time scale button and a second time scale button, the first time scale button and the second time scale button respectively correspond to the first specified time interval and a second specified time interval, and the method further comprises:
adjusting the medical information Gantt chart to present the specific medical information of the current patient of interest in the second specified time interval in response to determining that the second time scale button is triggered.

16. The patient information presentation method as claimed in claim 1, wherein the first patient information card comprises an examination item trend graph of the current patient of interest, wherein the examination item trend graph comprises a historical variation trend and a predicted variation trend of a specific examination item.

17. The patient information presentation method as claimed in claim 1, wherein the first patient information card comprises an early warning map of organ risk of the current patient of interest, wherein the early warning map of the organ risk is in form of a radar map, and the early warning map of the organ risk indicates a current risk score and a predicted risk score of each of a plurality of organ functions.

18. The patient information presentation method as claimed in claim 17, wherein in response to determining that the predicted risk score of a specific organ function of the plurality of organ functions exceeds a predetermined threshold, a warning prompt corresponding to the specific organ function is provided.

19. An electronic device, comprising:
a display, displaying a user interface;
a storage circuit, storing a program code;
an input interface, receiving a trigger event executed on the user interface; and
a processor, coupled to the display, the storage circuit, and the input interface, and the processor accesses the program code to:
switch the user interface to an editing interface in response to determining that an edit button in the user interface is triggered, wherein the editing interface comprises a first wallpaper and a plurality of patient information cards;
pin a first patient information card to a first position on the first wallpaper in a corresponding first predetermined size in response to determining that the first patient information card in the plurality of patient information cards is dragged to and placed at the first position on the first wallpaper; and
switch the user interface to a browsing interface in response to determining that an end editing button in the editing interface is triggered, and present the first wallpaper in the browsing interface, wherein the first wallpaper comprises the first patient information card set in the first position, and the first patient information card presents information corresponding to a current patient of interest.

20. The electronic device as claimed in claim 19, wherein after pinning the first patient information card to the first position on the first wallpaper in the corresponding first predetermined size, the processor is further configured to:
adjust a size of the first patient information card on the first wallpaper in response to the editing interface receiving a drag operation on an outer frame of the first patient information card.
